(19) United States
(12) Patent Application Publication
(10) Pub. No.: US 2022/0117263 A1
(43) Pub. Date: Apr. 21, 2022

(54) PET SUPPLEMENTS CONTAINING A CANNABINOID

(71) Applicant: American Alliance for Alternative and Natural Medicine, LLC, Augusta, GA (US)

(72) Inventor: Babak Baban, Augusta, GA (US)

(21) Appl. No.: 17/503,965

(22) Filed: Oct. 18, 2021

Related U.S. Application Data
(60) Provisional application No. 63/093,413, filed on Oct. 19, 2020.

(51) Int. Cl. A23K 10/16; A23K 20/111; A61K 35/741

(52) U.S. Cl. CPC A23K 10/16; A61K 35/741; A23K 20/111

(57) ABSTRACT

Disclosed herein are consumable products for pets such as dogs, cats, horses, and birds the consumable products containing a cannabinoid. The consumable products disclosed herein can be formulated as chews or liquid supplements with pleasing flavors in order to encourage the pets to eat the consumable products and derive therapeutic benefits from consumption. The consumable products contain live, active cultures of probiotic microorganisms or may contain spores of probiotic microorganisms. The consumable products have anti-inflammatory effects due to the presence of the cannabinoid but are non-narcotic. Further, the consumable products serve to enhance gut health due to the presence of probiotics. The consumable products are particularly useful for treating, preventing, and eliminating side effects from various diseases and conditions as well as side effects from medication use. In some aspects, the consumable products are co-administered or co-formulated with other therapeutic agents.

### CROSS-REFERENCE TO RELATED APPLICATIONS

This application claims the benefit of and priority to co-pending U.S. Provisional Patent Application No. 63/093,413, filed on Oct. 19, 2020, the contents of which are incorporated by reference herein in their entireties.

### BACKGROUND

Common domestic pets such as cats, dogs, birds, and horses, are hosts to several hundred different species of gut microorganisms, including bacteria, archaea, yeasts, and protozoa. Due to poor diet, medical treatments such as antibiotic use, and even stress or emotional factors, disruptions to the gut microbiota (known as “dysbiosis”) are increasingly common. Symptoms of dysbiosis range from gastrointestinal upset ranging from gas, bloating, and flatulence to indigestion or incomplete digestion, diarrhea, and constipation, fatigue, brittleness of the nails, hair loss, and more. Dysbiosis can be present in or on any part of the body but can be particularly problematic in the oral cavity and/or gastrointestinal tract and can have systemic effects, including effects on the nervous system.

Meanwhile, inflammation is part of the body's immune response and can be beneficial in response to an injury. In fact, the inflammatory response is required for wound healing and repairing damage to tissue. However, chronic inflammation (i.e., in the absence of any acute stimulus including, but not limited to, a wound or cut, a crush injury, a broken bone, and the like.) is increasingly a problem in modern society and has been linked to autoimmune disorders such as diabetes, inflammatory bowel disease, gum disease or periodontitis, osteoarthritis, allergic conditions including food sensitivities. Interest in controlling inflammation in pets is growing in both the veterinary community and the general population.

Dysbiosis and inflammation may be particularly problematic for pets undergoing treatment for cancer or other autoimmune conditions due to immunosuppressant effects of prescribed medications. Pets treated with chemotherapy and immunosuppressants also tend to experience side effects ranging from nausea, vomiting, nutrient deficiencies, and inappetence to constipation or diarrhea.

Temporary and long-term mood disorders including anxiety, stress, and depression can also affect pets. These can be triggered by changes to the environment such as a move or bringing a new pet into the home, dietary changes, loud noises, diagnostic procedures and treatment for a disease, and the like, or may be the result of a chemical imbalance in the nervous system.

It would be advantageous to have a single consumable product to address dysbiosis and inflammation as well as mood disorders and the side effects of chemotherapy and treatment for autoimmune disorders and other diseases. Further, it would be advantageous for the consumable product to have a pleasing taste so that the pet is encouraged to consume it without the need for stressful forced administration and for maximum reduction of inflammation, dysbiosis, mood disorders, and other medical conditions, as well as to maintain an optimal level of gut health.

### SUMMARY

Disclosed herein are consumable products for pets such as dogs, cats, horses, and birds, the consumable products containing a cannabinoid. The consumable products disclosed herein can be formulated as chews or liquid supplements with pleasing flavors in order to encourage the pets to eat the consumable products and derive therapeutic benefits from consumption. The consumable products contain live, active cultures of probiotic microorganisms or may contain spores of probiotic microorganisms. The consumable products have anti-inflammatory effects due to the presence of the cannabinoid but are non-narcotic. Further, the consumable products serve to enhance gut health due to the presence of probiotics. The consumable products are particularly useful for treating, preventing, and eliminating side effects from various diseases and conditions as well as side effects from medication use. In some aspects, the consumable products are co-administered or co-formulated with other therapeutic agents.

The advantages of the invention will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DETAILED DESCRIPTION

Before the present compounds, compositions, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an,” and “the” include plural referents unless the context clearly dictates otherwise. Thus, for example, reference to “a probiotic strain” includes mixtures of two or more probiotic strains, and the like.

Throughout this specification, unless the context dictates otherwise, the word “comprise,” or variations such as “comprises” or “comprising,” will be understood to imply the inclusion of a stated element, integer, step, or group of elements, integers, or steps, but not the exclusion of any other element, integer, step, or group of elements, integers, or steps.

“Optional” or “optionally” means that the subsequently described event or circumstance can or cannot occur, and that the description includes instances where the event or circumstance occurs and instances where it does not. For example, the phrase “optionally includes a natural flavor” means that the natural flavor may or may not be present.

As used herein, a plurality of items, structural elements, compositional elements, and/or materials may be presented in a common list for convenience. However, these lists should be construed as though each member of the list is individually identified as a separate and unique member. Thus, no individual member of any such list should be construed as a de facto equivalent of any other member of the same list based solely on its presentation in a common group, without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range was explicitly recited. As an example, a numerical range of “about 1” to “about 5” should be interpreted to include not only the explicitly recited values of about 1 to about 5, but also to include individual values and sub-ranges within the indicated range. Thus, included in this numerical range are individual values such as 2, 3, and 4, the sub-ranges such as from 1-3, from 2-4, from 3-5, from about 1 to about 3, from 1 to about 3, from about 1 to 3, etc., as well as 1, 2, 3, 4, and 5, individually. The same principle applies to ranges reciting only one numerical value as a minimum or maximum.

Furthermore, such an interpretation should apply regardless of the breadth or range of the characters being described.

References in the specification and concluding claims to parts by weight, of a particular element or component in a composition or article, denote the weight relationship between the element or component and any other elements or components in the composition or article for which a part by weight is expressed. Thus, in a composition containing 2 parts by weight of component X and 5 parts by weight of component Y, X and Y are present at a weight ratio of 2:5, and are present in such ratio regardless of whether additional components are contained in the composition.

A weight percent of a component, unless specifically stated to the contrary, is based on the total weight of the formulation or composition in which the component is included.

Disclosed are materials and components that can be used for, can be used in conjunction with, can be used in preparation for, or are products of the disclosed compositions and methods. These and other materials are disclosed herein, and it is understood that when combinations, subsets, interactions, groups, etc. of these materials are disclosed, that while specific reference to each various individual combination and permutation of these compounds may not be explicitly disclosed, each is specifically contemplated and described herein. For example, if a cannabinoid is disclosed and discussed, and a number of different probiotic strains are discussed, each and every combination of cannabinoid and probiotic strain that is possible is specifically contemplated unless specifically indicated to the contrary. For example, if a class of cannabinoids A, B, and C is disclosed, as well as a class of probiotic strains D, E, and F, and an example combination of A+D is disclosed, then even if each is not individually recited, each is individually and collectively contemplated. Thus, in this example, each of the combinations A+E, A+F, B+D, B+E, B+F, C+D, C+E, and C+F is specifically contemplated and should be considered from disclosure of A, B, ad C; D, E, and F; and the example combination A+D. Likewise, any subset or combination of these is also specifically contemplated and disclosed. Thus, for example, the sub-group of A+E, B+F, and C+E is specifically contemplated and should be considered from disclosure of A, B, and C; D, E, and F; and the example combination of A+D. This concept applies to all aspects of the disclosure including, but not limited to, steps in methods of making and using the disclosed compositions. Thus, if there are a variety of additional steps that can be performed with any specific embodiment or combination of embodiments of the disclosed methods, each such composition is specifically contemplated and should be considered disclosed.

The term “consumable product” as used herein is defined as an edible article such as, for example, a pet chew or liquid supplement, a pet treat such as, for example, a jerky treat, a freeze-dried treat, or a crunchy or soft treat, that is safe to be consumed by a dog, a cat, a horse, or a bird. In some aspects, the consumable product can be formulated differently (e.g., with different flavors added, or in different sizes) to appeal to a dog, cat, horse, or bird. In one aspect, the consumable product can be shaped in a common shape for a pet treat such as, for example, a bone shape for a dog biscuit or a fish shape for a cat treat, or any other shape (e.g., round, star-shaped, square shaped, rectangular, cubic, or the like). As used herein, a “chew” is a consumable product intended to be masticated and swallowed by the pet in the manner in which a pet treat or other food item might be consumed. Chews and other solid consumable products as disclosed herein can be soft or semi-moist or can be hard (e.g., baked, pressed into a tablet, or the like).

In some aspects, the consumable products can be formulated as a liquid. Further in this aspect, the liquid can be an oil, a water-based solution, a puree, a gel, or another form. In a still further aspect, liquids can be useful for dividing into smaller doses for animals with low body weight such as, for example, small birds (e.g., parakeets, finches, canaries, lovebirds, cockatiels, and the like). In an alternative aspect, liquids can be useful for providing to pets for whom pill administration is difficult. In one aspect, the liquid can be placed in the pet's water. In an alternative aspect, the liquid can be administered to the pet's mouth through a needle-less syringe, or can be placed on top of or mixed with the pet's food.

The term “treat” as used herein is defined as maintaining or reducing the symptoms of a pre-existing condition (e.g., inflammation) when compared to not consuming the consumable product. The term “prevent” as used herein is defined as eliminating or reducing the likelihood of the occurrence of one or more symptoms of a disease or disorder when compared to not consuming the consumable product (e.g. preventing one or more side effects of chemotherapy). The term “reduce” as used herein is defined as decreasing the severity of one or more symptoms (e.g., reducing the symptoms of a mood disorder, food allergy, gastrointestinal disorder, or the like) when compared to not consuming the consumable product.

Described herein are consumable products for pets comprising (a) a cannabinoid, (b) a probiotic, and (c) pumpkin, as well as other optional components. Each component used to produce the consumable products described herein and methods for producing and using the same are described below. In one aspect, although some benefits of cannabinoids, probiotics, pumpkin, and other components of the consumable products are individually known, the disclosed compositions are advantageous for several reasons. In another aspect, combining beneficial ingredients in one consumable product requires pet owners, veterinarians, and others to administer fewer individual medications or supplements to pets simultaneously, which can be especially important to animals resistant to the administration of medications. Further in this aspect, texture-modifying additives and flavors can make the consumable products more appealing to animals, such that they willingly consume the disclosed products.

In another aspect, combining beneficial ingredients in one consumable product can have an additive effect such as, for example, addressing systemic inflammation over time through improvement of gastrointestinal bacterial balance while also addressing the effects of inflammation such as, for example, arthritis pain using a cannabinoid. In still another aspect, combining beneficial ingredients in one consumable product can address two different conditions with only one consumable product described herein; for example, treating anxiety resulting from illness and subsequent vet visits with a cannabinoid while also treating diarrhea with a probiotic and pumpkin. In some aspects, combining beneficial ingredients in one consumable product can address different symptoms of the same condition simultaneously; for example, a cannabinoid can reduce inflammation from inflammatory bowel disease while probiotics and pumpkin can function synergistically to improve the gut microbiome and reduce or lessen the severity of episodes of diarrhea.

In one aspect, described herein are consumable products for pets with a pleasant flavor and texture. In a further aspect, the flavor and texture may be useful in enhancing or encouraging pet consumption of the products and/or compliance with a treatment regimen that includes the products. In another aspect, the consumable products can be formulated as hard chews, soft chews, treats (e.g., dog biscuits, jerky treats, soft or hard treats, tablets, and the like) and/or liquid supplements.

The consumable product can be prepared in any weight as desired. In one aspect, the serving size of the consumable product is from about 1 gram to about 10 grams, from 2 grams to about 3 grams, or is about 1, 1.5, 2, 2.5, 3, 3.5, 4, 4.5, 5, 5.5, 6, 6.5, 7, 7.5, 8, 8.5, 9, 9.5, or about 10 grams, or a combination of any of the foregoing values, or a range encompassing any of the foregoing values. In some aspects, when the consumable products are liquids, the consumable products do not have a set weight but are provided in a multi-dose bottle with a graduated dispenser or dropper or instructions for an amount to administer.

Cannabinoid

The consumable products described herein contain one or more cannabinoids. Cannabinoids are a class of molecules produced by cannabis plants; over one hundred of these have been identified thus far. The three most abundant cannabinoids include cannabidiol (CBD), tetrahydrocannabinol (THC), and cannabinodiol (CBND). Although some cannabinoids, such as THC, are psychoactive, others (i.e., CBD) are not. In one aspect, the cannabinoid includes tetrahydrocannabinol, tetrahydrocannabinolic acid, cannabidiol, cannabidiolic acid, cannabinol, cannabigerol, cannabichromene, cannabicyclol, cannabivarin, tetrahydrocannabivarin, cannabidivarin, cannabichromevarin, cannabigerovarin, cannabigerol monomethyl ether, cannabielsoin, cannabicitran, or any combination thereof. In another aspect, the cannabinoid is cannabidiol.

In one aspect, the consumable product can be administered to a pet such that the pet receives from about 1 to about 5 mg of cannabinoid per 10 pounds of pet weight, or about 1, 1.5, 2, 2.5, 3, 3.5, 4, 4.5, or about 5 mg per 10 pounds of pet weight, or a combination of any of the foregoing values, or a range encompassing any of the foregoing values. In some aspects, for example with medium-sized and large dogs, or with horses, more than one unit (e.g., more than one chew, treat, etc.) can be administered to achieve the desired dosage level for the cannabinoid. In other aspects, larger solid products can be formulated for larger animals. In other aspects, a chew, treat, tablet, or the like can be broken or split in order to provide a lower dosage level for a milder condition, small or toy dog, or a cat. In some aspects, dosage can be tapered over time. Further in this aspect, it may be advisable in certain circumstances to start at a lower dose per 10 pounds of body weight (e.g., 1 mg per 10 pounds) and increase to the maximum tolerated dose. In other aspects, if the condition being treated is temporary or resolves, a higher dosage can be reduced to a lower maintenance level or eliminated over time. In an alternative aspect, a liquid formulation such as an oil or gel can have a sliding dosage scale based on body weight of the animal to which it is being administered.

Probiotic

The consumable products disclosed herein include a probiotic. In one aspect, the probiotic is a live microorganism that can colonize in the digestive system. In another aspect, the probiotic is a microbial spore that is shelf stable and can survive the harsh environment of the upper gastrointestinal tract in order to establish a population in the gut.

In one aspect, the probiotic is a yeast such as, for example, an organism selected from the genus Saccharomyces, Debaromyces, Candida, Pichia, or Torulopsis. In an alternative aspect, the probiotic is a mold such as, for example, an organism selected from the genus Aspergillus, Rhizopus, Mucor, or Penicillium. In still another aspect, the probiotic is a bacterium such as, for example, an organism selected from the genus Bifidobacterium, Bacterioides, Clostridium, Fusobacterium, Melissococcus, Propionibacterium, Streptococcus, Enterococcus, Lactococcus, Kocuria, Staphylococcus, Peptostreptococcus, Bacillus, Pediococcus, Micrococcus, Euconostoc, Weissella, Aerococcus, Oenococcus, or Lactobacillus. In still another aspect, the probiotic is a combination of organisms from one or more of the genera listed above.

In another aspect, the probiotic is Aspergillus niger, A. oryzae, Bacillus coagulans, B. lentus, B. licheniformis, B. mesentericus, B. pumilus, B. subtilis, B. natto, Bacteroides amylophilus, Bac. capillosus, Bac. ruminocola, Bac. suis, Bifidobacterium adolescentis, Bif animalis, Bif breve, Bif bifidum, Bif infantis, Bif. lactis, Bif. longum, Bif pseudolongum, Bif thermophilum, Candida pintolepesii, Clostridium butyricum, Enterococcus cremoris, E. diacetylactis, E. faecium, E. intermedius, E. lactis, E. mundtii, E. thermophilus, Escherichia coli, Kluyveromyces fragilis, Lactobacillus acidophilus, L. alimentarius, L. amylovorus, L. crispatus, L. bifidus, L. brevis, L. bulgaricus, L. casei, L. curvatus, L. cellobiosus, L. delbrueckii ss. bulgaricus, L. farciminis, L. fermentum, L. gasseri, L. helveticus, L. lactis, L. plantarum, L. johnsonii, L. paracasei, L. reuteri, L. rhamnosus, L. sakei, L. salivarius, Leuconostoc mesenteroides, Pediococcus cereviseae (damnosus), P. acidlactici, P. pentosaceus, Propionibacterium freudenreichii, Prop. shertnanii, Saccharomyces cereviseae, Staphyloccus carnosus, Staph. xylosus, Streptococcus infantarius, Strep. salivarius ss. thermophilus, Strep. thermophilus, Strep. lactis, a fermentation product thereof, or any combination thereof.

In one aspect, the probiotic can be a combination of one or more of Bacillus coagulans GBI-30, 6086 and/or another Bacillus coagulans strain; Lactobacillus acidophilus; Lactobacillus plantarum; Lactobacillus brevis; Lactobacillus fermentum; and Lactobacillus lactis.

As used herein, a “colony forming unit” or “CFU” is a measure of how many bacteria in a sample are capable of forming colonies. In this aspect, microorganisms are cultured and only viable cells are counted as CFUs. In one aspect, it is uncertain if a given colony on a culture plate arose from one cell or a group of several cells, hence the use of CFUs to address the uncertainty.

In one aspect, the consumable product has from about 1 to about 10 billion colony forming units (CFUs) of probiotic organisms per unit dose of consumable product, or about 1, 2, 3, 4, 5, 6, 7, 8, 9, or about 10 billion CFUs of probiotic organisms per unit dose of consumable product. In still another aspect, the consumable product has 3 billion CFUs of probiotic organisms per unit dose of consumable product. In one aspect, all of the CFUs are from the same organism. In an alternative aspect, the CFUs are from a mixture of organisms.

Pumpkin

In one aspect, pumpkin is used to produce the consumable products described herein. In a further aspect, the pumpkin can be dehydrated, powdered, baked, pureed, or processed by any means typically used to process pumpkin. In some aspects, the pumpkin can contain added water for processing purposes. In other aspects, a pumpkin puree contains no added liquids.

The amount of pumpkin incorporated into the consumable product can vary depending upon the desired taste and/or texture of the product as well as on the desired therapeutic effect of the pumpkin.

In one aspect, the consumable product can be administered to a pet such that the pet receives from about 1 to about 5 mg of pumpkin per 10 pounds of pet weight, or about 1, 1.5, 2, 2.5, 3, 3.5, 4, 4.5, or about 5 mg per 10 pounds of pet weight, or a combination of any of the foregoing values, or a range encompassing any of the foregoing values. In some aspects, for example with medium-sized and large dogs, or with horses, more than one chew or solid consumable product, or a higher volume of liquid consumable product, can be administered to achieve the desired dosage level for the pumpkin.

In another aspect, the pumpkin can function as a prebiotic. As used herein, “prebiotic” refers to a food ingredient that promotes the growth of beneficial microorganisms (such as, for example, microorganisms consumed as probiotics) in the intestine. In addition to supporting the growth of probiotic organisms, a prebiotic can provide an additional benefit such as, for example, being a source of fiber, which can assist with the transit of food through the digestive tract. In some aspects, prebiotics can assist with the resolution of diarrhea and/or constipation.

Additional Prebiotics

In another aspect, the consumable product can contain one or more prebiotic ingredients in addition to the pumpkin. In addition to their prebiotic benefits, in some aspects, the prebiotic ingredients can contribute to the texture, mouth feel, or taste of the consumable products, or can assist with binding the other ingredients of the consumable product together.

In one aspect, the prebiotic can be a fiber. In one aspect, the prebiotic is a fiber derived from psyllium husk, beet pulp, or another fiber source. Using techniques known in the art, the fiber can be separated and isolated from the natural sources listed above. In another aspect, the prebiotic can be a fructooligosaccharide. A “fructooligosaccharide” or “FOS” or “oligofructose” is a fructan with a short chain length. As an example, the degree of polymerization in inulin can be from 10 to 60, or in some instances, can be up to several thousand fructose units, whereas the degree of polymerization in FOS produced from the chemical degradation of inulin can be from 1 to 14 or from 1 to 7. FOS may improve the flavor or odor of food products; FOS is often used as alternative sweeteners, but FOS, like inulin, can serve as a substrate for probiotic microorganisms in the large intestine.

In another aspect, the consumable products can contain another type of prebiotic including, but not limited to, pectin, β-glucan, guar gum, methylcellulose, resistant starch, or any combination thereof. In still another aspect, the consumable products contain a combination of soluble and insoluble fiber.

In one aspect, the consumable products described herein are “symbiotic.” This term refers to products containing both probiotics and prebiotics and specifically to products wherein the prebiotics are chosen selectively to favor the growth of the probiotic microorganisms.

Natural Preservative

A “preservative” is a compound or composition that is added to food, beverage, and/or other consumable products to prevent spoilage and prolong shelf life. In one aspect, a preservative can be a natural preservative. In one aspect, the natural preservative can be selected from rosemary extract, sorbic acid, mixed tocopherols or vitamin E, green tea extract, or any combination thereof. In another aspect, the consumable products described herein do not contain any added or artificial preservatives.

In one aspect, the natural preservative is rosemary extract. In another aspect, the rosemary extract is provided in an amount of from about 1% to 3% per unit dose of the consumable product. In a further aspect, for a liquid supplement, the rosemary extract is provided in an amount of from about 1% to about 3% (v/v), or at about 1, 1.5, 2, 2.5, or about 3% (v/v), or a combination of any of the foregoing values, or a range encompassing any of the foregoing values. In an alternative aspect, for a solid supplement (e.g., treat or chew), the rosemary extract is provided in an amount of from about 1% to about 3% (w/w), or at about 1, 1.5, 2, 2.5, or about 3% (w/w), or a combination of any of the foregoing values, or a range encompassing any of the foregoing values.

Flavoring

In one aspect, the consumable products include one or more natural flavors. In a further aspect, the flavors appeal to pets and can entice pets to eat the consumable products without requiring forceful administration of the consumable products, which may lead to stress and/or anxiety.

In another aspect, the natural flavor can be selected from bacon, chicken, turkey, duck, lamb, beef liver, chicken liver, dairy, salmon, tuna, scallop, shrimp, dried brewer's yeast, cinnamon, peanut butter, apples, blueberries, sweet potato, molasses, tapioca syrup, dried milk, dried egg whites, or any combination thereof.

In some aspects, the natural flavor can be different for different animals. Further in this aspect, in one example, peanut butter and molasses are examples of natural flavors that may appeal to dogs, while cats may prefer fish or seafood flavors. In another aspect, horses may prefer fruit or vegetable flavors (e.g., apple or carrot), while birds may prefer fruit flavors, nut flavors, vegetable flavors, and the like. In other aspects, the natural flavors can vary based on some other factor such as, for example, for the sake of providing variety and stimulation to the pet, or for the purpose of distinguishing versions of the consumable product with different therapeutic effects (e.g., a consumable product that helps with joint pain and inflammation can be flavored with one combination of natural flavors such as, for example, chicken and bacon, while a consumable product that treats the symptoms of inflammatory bowel disease can be formulated with hypoallergenic ingredients including, but not limited to, flavors such as duck sourced from novel proteins that will not irritate the gastrointestinal tract of the animal being treated).

Other Ingredients

Texture-Modifying Agents

In one aspect, the consumable products disclosed herein can include one or more texture-modifying agents in addition to or instead of the prebiotics disclosed herein. In a further aspect, the texture-modifying agent is a food-grade ingredient that can change the texture (e.g., hard or soft chew, chewable tablet, dog biscuit, oil, liquid supplement, etc.), mouth feel, or another property of the consumable product (e.g., binding the ingredients together into a uniformly-sized whole unit dose), such that the consumable product is appealing to a cat, dog, horse, or bird.

In another aspect, the texture-modifying agent can include, but is not limited to, glycerin, polysorbate 80, flaxseed or flaxseed oil, medium chain triglyceride oil, oat flour, palm fruit oil, powdered cellulose, white rice flour, brown rice flour, rice bran oil, soybean oil, sunflower lecithin, coconut oil, rolled oats, potato starch, peas, gum arabic, sodium alginate, salmon oil, anchovy oil, sardine oil, or another fish oil, wheat germ oil, malic acid, propylene glycol, calcium sulfate dehydrate, dextrin, purified water, or any combination thereof

Additional Active Ingredients

In another aspect, the consumable products disclosed herein include at least one additional active ingredient. In another aspect, the one additional active ingredient can provide additional support for the condition, disease, and/or symptoms being experienced by the pet, or which the owner of the pet desires to prevent. In another aspect, the at least one additional active ingredient is selected from components useful for joint support (e.g. glucosamine hydrochloride, methylsulfonylmethane, chondroitin sulfate, bone broth, collagen), components useful for reducing inflammation and/or for protection of skin and coat (e.g., fish oil, ginger, turmeric, Boswellia), components useful for relieving anxiety or promoting sleep (e.g., chamomile, passionflower, L-tryptophan, L-theanine, lemon balm, valerian), sources of essential fatty acids and/or proteins (e.g., hempseed powder, hempseed oil), components useful for liver support (e.g., milk thistle extract, S-adenosylmethionine), sources of antioxidants (e.g., seaweed, chlorella), nutritional supplements (e.g., β-carotene, taurine, a vitamin, a mineral), medicinal herbs (e.g., tea tree oil, slippery elm, aloe vera, dandelion), an electrolyte (e.g., rock salt, potassium chloride, magnesium sulfate, calcium lactate, potassium sulfate) or any combination thereof.

Method for Making a Consumable Product

In one aspect, to produce a consumable product according to the present disclosure, all ingredients are combined. In a further aspect, combined ingredients can be placed in a tank connected to a canning machine. In a still further aspect, the mixture can be forced or extruded through an opening and onto a metal sheet. In one aspect, when placed on the metal sheet, the mixture can have a thickness of from about 8 mm to about 12 mm, or of about 8, 9, 10, 11, or 12 mm, or a combination of any of the foregoing values, or a range encompassing any of the foregoing values. In one aspect, the extruded mixture can then be heated thoroughly to cook the ingredients. In a further aspect, cooking can be performed in an oven, in a microwave, or through steam heating. In one aspect, the sheet containing the mixture can be heated by a source that provides heat to both the top and the bottom of the mixture. In any of these aspects, the internal temperature of the mixture can be heated to at least about 77° C. In one aspect, once cooked, the mixture can be directly cut into chews, jerky, dog biscuits, or another format, or can be pulverized for dissolution in a carrier liquid. In some aspects, the metal sheet can be formed as a mold to produce the consumable product in solid form (e.g. in the shape of a dog bone) without the need for subsequent cutting and producing less wasted product during the manufacturing process.

Method for Reducing Inflammation

In one aspect, the consumable products described herein are useful for treating, preventing, or reducing inflammation. “Inflammation” as used herein is part of the body's response to harmful stimuli. In the instance of injury or tissue damage, inflammation is a protective response localized to a specific area (such as, for example, redness and swelling around a burn). In another aspect, inflammation can be chronic inflammation or systemic inflammation. Further in this aspect, chronic or systemic inflammation can contribute to an overall decline in the health of the body.

In one aspect, the inflammation can be produced by chemotherapy or cancer treatment. Depending upon the type of cancer that is treated and the chemotherapeutic agent selected, inflammation can result in different parts of the pet experiencing cancer. In another aspect, the inflammation is produced by cancer present in the pet. Inflammation has been linked to several different types of cancer.

In another aspect, the consumable products described herein can reduce or prevent inflammation caused by a gastrointestinal disorder, an infection, or an autoimmune disorder. In one aspect, consumable products described herein can reduce or prevent inflammation caused by or resulting in inflammatory bowel disease, diabetes, obesity, dental disease, allergies, asthma, food sensitivities, cardiovascular disease, yeast infection, bacterial infection, viral infection, parasitic infection, diabetes, or any combination thereof.

In one aspect, disclosed herein is a method for treating, preventing, or reducing inflammation in a pet, the method including the step of administering to the pet the disclosed consumable product.

Method for Correcting or Improving Cases of Dysbiosis

In another aspect, consumption of the products described herein can correct or improve cases of dysbiosis. As used herein, “dysbiosis” refers to an imbalance of the microbial population on or inside the body, such as in the intestinal tract. In some aspects, when a pet is in a state of dysbiosis, pathogenic bacteria, viruses (e.g., feline herpesvirus, calicivirus, and the like), and other parasites (e.g., Demodex mites in dogs) normally present in low amounts flourish. This can lead to improper digestion, malabsorption of food and/or specific nutrients, and other illnesses, including, but not limited to, yeast infections of the skin and/or ears, mange, ulcerations of the eye, and the like. In some aspects, medical conditions can be exacerbated due to competition between pathogenic bacteria and useful bacteria such as, for example, those that produce certain vitamins (e.g., vitamin K) from dietary precursors. In another aspect, accumulation of waste products from pathogenic bacteria is another harmful effect of dysbiosis. Prolonged dysbiosis can result from inappropriate diet and/or dietary indiscretions, antibiotic exposure, conditions such as inflammatory bowel disease, and the like. Dysbiosis in the intestinal tract is best known, but a state of dysbiosis can also be found among skin flora. In one aspect, consumption of the products described herein leads to a reduction or improvement in a case of dysbiosis. In one aspect, the dysbiosis can be a result of chemotherapy or cancer treatment, or prolonged steroid exposure for treatment of an autoimmune disease, asthma, or the like. In an alternative aspect, the dysbiosis can be from another source.

In one aspect, disclosed herein is a method for treating, preventing, or reducing dysbiosis in a pet, the method including the step of administering to the pet the disclosed consumable product.

Method for Treating, Preventing, or Reducing at Least One Symptom of a Disease or Condition in a Pet

In one aspect, provided herein is a method for treating, preventing, or reducing one or more symptoms of a disease or condition in a pet, the method including at least the step of administering the consumable product disclosed herein to the pet.

In one aspect, one serving of the consumable product should be eaten per day. In another aspect, a subject can eat one serving of the consumable product at one or more scheduled times each day, such as, for example, one hour or 30 minutes prior to a meal, or with a meal.

In any of these aspects, the disease or condition can include cancer, osteoarthritis, epilepsy, inflammatory bowel disease, dental disease, cardiovascular disease, diabetes, obesity, mood disorders, autoimmune disease, yeast infection, bacterial infection, viral infection, parasitic infection, anxiety, dysbiosis, allergies, food sensitivities, asthma, or a combination thereof. Further in this aspect, the one or more symptom can include pain, appetite loss, noise phobia, nausea, inflammation, diarrhea, constipation, gas, bloating, bad breath, leaky gut, a nutrient deficiency, stress, medication side effects, or a combination thereof.

In some aspects, the consumable product can be co-administered to the pet along with at least one other medication effective against the disease or condition. In another aspect, the at least one other medication can be selected from an antibiotic (e.g. gentamicin, tobramycin, amikacin, streptomycin, kanamycin, penicillin G, penicillin V, penicillinase-resistant penicillin, methicillin, oxacillin, nafcillin, extended-spectrum penicillin, ampicillin, amoxicillin, carbenicillin, cephalothin, cefamandole, cefataxime, primaxin, aztreonam, chloramphenicol, lincomycin, clindamycin, pirlimycin, tylosin, erythromycin, sulfadiazine, sulfamethoxazole, sulfadoxine, chlortetracycline, oxytetracycline, demethylchlortetracycline, rolitetracycline, lymecycline, clomocycline, methacycline, doxycycline, minocycline), a chemotherapy drug (e.g. cyclophosphamide, chlorambucil, lomustine, dacarbazine, ifosfamide, doxorubicin, mitoxantrone, actinomycin D, methotrexate, cytosine arabinoside, vinblastine, vincristine, asparaginase, carboplatin, cisplatin, procarbazine, bleomycin, 1-asparaginase, melphalan, mitoxantrone), a steroid (prednisone, prednisolone, cortisol, methylprednisolone, fludrocortisone, isoflupredone, triamcinolone, dexamethasone, betamethasone, flumethasone), an antifungal medication (e.g. Ketoconazole, itraconazole, voriconazole, fluconazole, posaconazole, terbinafine, griseofulvin, potassium iodide), an anti-parasitic agent (e.g., ivermectin, moxidectin, thiabendazole, mebendazole, fenbendazole, cambendazole, oxibendazole, flubendazole, oxfendazole, albendazole, pyrantel, morantel, N-butyl chloride), a painkiller or analgesic (e.g. carprofen, deracoxib, firocoxib, meloxicam, piroxicam, robenacoxib, tramadol, codeine, hydrocodone, morphine, hydromorphone, buprenorphine, amantadine, gabapentin, oxcarbazepine, amitriptyline), an antidepressant (fluoxetine, buspirone, diazepam, oxazepam, acepromazine, chlorpromazine, thioridazine, haloperidol, fluphenazine, trifluoperazine, prochlorperazine, thiothixene, risperidone, carbamazepine, lithium, amitriptyline, imipramine, clomipramine, doxepine, sertraline, paroxetine, selegiline), an antihistamine (e.g. diphenhydramine, cetirizine, chlorpheniramine, hydroxyzine, eicosapentaenoic acid), an immunosuppressive agent (e.g. azathioprine, cyclosporine, leflunomide, mycophenolate, anti-cancer agents and corticosteroids already mentioned), an anticonvulsant (e.g. phenobarbital, primidone, diazepam, potassium bromide, phenytoin, valproic acid, zonisamide, levetiracetam, felbamate), an appetite stimulant (e.g. mirtazapine, megestrol acetate, cyproheptadine, capromorelin oral solution), an antiemetic (e.g. acepromazine, chlorpromazine, prochlorperazine, aminopentamide, dimenhydrinate, butorphanol, metoclopramide, ondansetron, granisetron, maropitant), an appetite suppressant (e.g. dirlotapide), or a combination thereof. In some aspects, the consumable product and the at least one other medication can be co-formulated. In one aspect, a veterinarian may prescribe the at least one other medication to address a medical condition being experienced by the pet and prescribe or suggest to the owner that the pet also take the disclosed consumable product to help with one or more side effects of the other medication and/or to provide additional support in dealing with the disease, condition, or symptom being experienced by the pet.

In some aspects, administration of the consumable product to the pet can prevent or reduce the severity of at least one side effect of a medication (e.g., can reduce nausea or inappetence resulting from the administration of an analgesic, can reduce dysbiosis resulting from the administration of an antibiotic, or the like).

Aspects

Aspect 1: A consumable product for a pet comprising (a) a cannabinoid, (b) a probiotic, and (c) pumpkin.

Aspect 2: The consumable product of Aspect 1, wherein the consumable product comprises a solid or a liquid.

Aspect 3: The consumable product of Aspect 2, wherein the solid comprises a hard chew, a soft chew, a biscuit, a freeze-dried treat, or a jerky treat.

Aspect 4: The consumable product of Aspect 2 or 3, wherein the solid has a weight of from about 1 g to about 10 g.

Aspect 5: The consumable product of any of Aspects 1-4, wherein the cannabinoid comprises tetrahydrocannabinol, tetrahydrocannabinolic acid, cannabidiol, cannabidiolic acid, cannabinol, cannabigerol, cannabichromene, cannabicyclol, cannabivarin, tetrahydrocannabivarin, cannabidivarin, cannabichromevarin, cannabigerovarin, cannabigerol monomethyl ether, cannabielsoin, cannabicitran, or a combination thereof.

Aspect 6: The consumable product of Aspect 5, wherein the cannabinoid is cannabidiol.

Aspect 7: The consumable product of any of Aspects 1-6, wherein the probiotic comprises Bacillus coagulans GBI-30, 6086 or another Bacillus coagulans strain; Lactobacillus acidophilus; Lactobacillus plantarum; Lactobacillus brevis; Lactobacillus fermentum; Lactobacillus lactis; or any combination thereof.

Aspect 8: The consumable product of any of Aspects 1-7, wherein the probiotic is present in an amount of from about 1 to about 10 billion colony-forming units per unit dose.

Aspect 9: The consumable product of any of Aspects 1-7, wherein the probiotic is present in an amount of about 3 billion colony-forming units per unit dose.

Aspect 10: The consumable product any of Aspects 1-8, further comprising a natural preservative.

Aspect 11: The consumable product of Aspect 10, wherein the natural preservative comprises rosemary extract, sorbic acid, mixed tocopherols, green tea extract, or a combination thereof.

Aspect 12: The consumable product of Aspect 11, wherein the consumable product comprises from about 1% to about 3% by volume of liquid consumable product or from about 1% by 3% by weight of solid consumable product.

Aspect 13: The consumable product of Aspect 11 or 12, wherein the natural preservative comprises rosemary extract.

Aspect 14: The consumable product of any of Aspects 1-13, wherein the consumable product does not include any artificial preservatives.

Aspect 15: The consumable product of any of Aspects 1-14, wherein the product further comprises a prebiotic.

Aspect 16: The consumable product of Aspect 15, wherein the prebiotic comprises pectin, β-glucan, guar gum, methylcellulose, psyllium fiber, resistant starch, fructooligosaccharides, beet pulp, or any combination thereof.

Aspect 17: The consumable product of any of Aspects 1-16, wherein the consumable product further comprises a natural flavor.

Aspect 18: The consumable product of Aspect 17, wherein the natural flavor comprises bacon, chicken, turkey, duck, lamb, beef liver, chicken liver, dairy, salmon, tuna, scallop, shrimp, dried brewer's yeast, cinnamon, peanut butter, apples, blueberries, sweet potato, molasses, tapioca syrup, dried milk, dried egg whites, or any combination thereof.

Aspect 19: The consumable product of any of Aspects 1-18, wherein the consumable product further comprises a texture-modifying agent.

Aspect 20: The consumable product of Aspect 19, wherein the texture-modifying agent comprises glycerin, polysorbate 80, flaxseed or flaxseed oil, medium chain triglyceride oil, oat flour, palm fruit oil, powdered cellulose, white rice flour, brown rice flour, rice bran oil, soybean oil, sunflower lecithin, coconut oil, rolled oats, potato starch, peas, gum arabic, sodium alginate, salmon oil, anchovy oil, sardine oil, or another fish oil, wheat germ oil, malic acid, propylene glycol, calcium sulfate dehydrate, dextrin, purified water, or any combination thereof.

Aspect 21: The consumable product of any of Aspects 1-20, wherein the consumable product further comprises at least one additional active ingredient.

Aspect 22: The consumable product of Aspect 21, wherein the at least one additional active ingredient comprises glucosamine hydrochloride, methylsulfonylmethane, chondroitin sulfate, fish oil, chamomile, passionflower, ginger, L-tryptophan, turmeric, Boswellia, hempseed powder, hempseed oil, bone broth, collagen, milk thistle extract, chamomile, lemon balm, valerian, turmeric, tea tree oil, slippery elm, aloe vera, dandelion, S-adenosylmethionine, L-theanine, seaweed, chlorella, β-carotene, taurine, a vitamin, a mineral, an electrolyte, or any combination thereof.

Aspect 23: The consumable product of any of Aspects 1-22, wherein the consumable product comprises:
a. cannabidiol in an amount such that about 1 mg to about 5 mg are administered per 10 pounds of pet body weight;
b. a probiotic comprising about 1 to about 10 billion colony forming units of Bacillus coagulans GBI-30, 6086 or another Bacillus coagulans strain; Lactobacillus acidophilus; Lactobacillus plantarum; Lactobacillus brevis; Lactobacillus fermentum; Lactobacillus lactis; or any combination thereof and
c. pumpkin in an amount such that about 3 mg are administered per 10 pounds of pet body weight.

Aspect 24: The consumable product of any of Aspects 1-23, wherein the pet is a cat, a dog, a horse, or a bird.

Aspect 25: A method for making the consumable product of any of the preceding Aspects, the method comprising:
a. mixing the cannabinoid, probiotic, pumpkin, and optionally one or more additional ingredients to produce a mixture;
b. extruding the mixture; and
c. heating the mixture to produce a cooked mixture.

Aspect 26: The method of Aspect 25, wherein the mixture following step (b) comprises a thickness of from about 8 to about 12 mm.

Aspect 27: The method of Aspect 25 or 26, wherein heating the mixture is accomplished in an oven, in a microwave, or through steam heating.

Aspect 28: The method of any of Aspects 25-27, wherein heating the mixture causes the mixture to reach an internal temperature of at least about 77° C.

Aspect 29: The method of any of Aspects 25-28, further comprising cutting the cooked mixture into pieces.

Aspect 30: The method of any of Aspects 25-28, wherein, in step (b), the mixture is extruded into a shaped mold.

Aspect 31: The method of any of Aspects 25-28, further comprising:
d. pulverizing the cooked mixture to form a powder; and
e. mixing the powder with a carrier liquid to produce a liquid consumable product.

Aspect 32: A method for treating, preventing, or reducing at least one symptom of a disease or condition in a pet, the method comprising administering the consumable product of any of Aspects 1-24 or a consumable product made according to the method of any of Aspects 25-31 to the pet.

Aspect 33: A method for reducing or preventing at least one side effect of a medication on a pet, the method comprising administering the consumable product of any of Aspects 1-24 or a consumable product made according to the method of any of Aspects 25-31 to the pet.

Aspect 34: The method of Aspect 32 or 33, wherein the consumable product is administered in a dosage of from about 1 to about 5 mg of cannabinoid per 10 pounds of pet body weight.

Aspect 35: The method of Aspect 32 or 33, wherein the consumable product is administered in a dosage of about 3 mg of pumpkin per 10 pounds of pet body weight.

Aspect 36: The method of any of Aspects 32-35, wherein the disease or condition comprises cancer, osteoarthritis, epilepsy, inflammatory bowel disease, dental disease, cardiovascular disease, diabetes, obesity, mood disorders, autoimmune disease, yeast infection, bacterial infection, viral infection, parasitic infection, anxiety, dysbiosis, allergies, food sensitivities, asthma, or a combination thereof.

Aspect 37: The method of any of Aspects 32-36, wherein at least one symptom or the at least one side effect comprises pain, appetite loss, noise phobia, nausea, inflammation, diarrhea, constipation, gas, bloating, bad breath, leaky gut, a nutrient deficiency, stress, or a combination thereof.

Aspect 38: The method of any of Aspects 32-37, further comprising co-administering the consumable product with at least one other medication effective against the disease or condition.

Aspect 39: The method of Aspect 38, wherein the at least one other medication comprises an antibiotic, a chemotherapy drug, a steroid, an antifungal medication, an anti-parasitic agent, a painkiller or analgesic, an antidepressant, an antihistamine, an immunosuppressive agent, an anticonvulsant, an appetite stimulant, an antiemetic, an appetite suppressant, or a combination thereof.

Aspect 40: The method of Aspect 38 or 39, wherein the consumable product and the at least one other medication are co-formulated.

### EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperatures, etc.) but some errors and deviations should be accounted for. Unless indicated otherwise, parts are parts by weight, temperature is in ° C. or is at ambient temperature, and pressure is at or near atmospheric. Numerous variations and combinations of reaction conditions, e.g., component concentrations, desired solvents, solvent mixtures, temperatures, pressures, and other reaction ranges and conditions can be used to optimize the product purity and yield obtained from the described process. Only reasonable and routine experimentation will be required to optimize such processes and conditions.

In one aspect, the consumable product disclosed herein includes the following components:

TABLE 1
Ingredients of Consumable Product, Per Individual Serving

| Component | Amount |
|---|---|
| Cannabidiol | 1-5 mg/10 pounds of pet body weight |
| Probiotic culture^{a} | 3 billion CFU/unit dose |
| Pumpkin | 3 gram/10 pounds of pet body weight |
| Rosemary extract | 1-3% of unit dose |
| Flavoring | 3 mL/64 ounces (liquid supplement) or 2 gram/ |
| | 85 gram (solid treat or chew) |

^{a}In one example formulation, the probiotic culture consists of Bacillus coagulans GBI-30, 6086 or another Bacillus coagulans strain; Lactobacillus acidophilus; Lactobacillus plantarum; Lactobacillus brevis; Lactobacillus fermentum; and Lactobacillus lactis.

To produce a consumable product according to the present disclosure, all ingredients are combined and placed in a tank connected to a canning machine. The mixture is forced through an opening and onto a metal sheet such that the mixture has a thickness of from 8 to 12 mm. The mixture is heated thoroughly to cook the ingredients; this has been performed in an oven, in a microwave, or through steam heating. The sheet containing the mixture is typically heated by a source that can provide heat to both the top and the bottom of the mixture, allowing the internal temperature to reach a minimum of 77° C. Once cooked, the mixture can be directly cut into chews, biscuits, or other treat shapes, or can be pulverized for dissolution into a carrier liquid.

Throughout this application, various publications are referenced. The disclosures of these publications in their entireties are hereby incorporated by reference into this application in order to more fully describe the compounds, compositions, and methods described herein.

Various modifications and variations can be made to the compounds, compositions, and methods described herein. Other aspects of the compounds, compositions, and methods described herein will be apparent from consideration of the specification and practice of the compounds, compositions, and methods disclosed herein. It is intended that the specification and examples be considered as exemplary.

What is claimed:

1. A consumable product for a pet comprising (a) a cannabinoid, (b) a probiotic, and (c) pumpkin.

2. The consumable product of claim 1, wherein the consumable product comprises a solid or a liquid.

3. The consumable product of claim 2, wherein the solid comprises a hard chew, a soft chew, a biscuit, a freeze-dried treat, or a jerky treat.

4. The consumable product of claim 2, wherein the solid has a weight of from about 1 g to about 10 g.

5. The consumable product of claim 1, wherein the cannabinoid comprises tetrahydrocannabinol, tetrahydrocannabinolic acid, cannabidiol, cannabidiolic acid, cannabinol, cannabigerol, cannabichromene, cannabicyclol, cannabivarin, tetrahydrocannabivarin, cannabidivarin, cannabichromevarin, cannabigerovarin, cannabigerol monomethyl ether, cannabielsoin, cannabicitran, or a combination thereof.

6. The consumable product of claim 5, wherein the cannabinoid is cannabidiol.

7. The consumable product of claim 1, wherein the probiotic comprises Bacillus coagulans GBI-30, 6086 or another Bacillus coagulans strain; Lactobacillus acidophilus; Lactobacillus plantarum; Lactobacillus brevis; Lactobacillus fermentum; Lactobacillus lactis; or any combination thereof.

8. The consumable product of claim 1, wherein the probiotic is present in an amount of from about 1 to about 10 billion colony-forming units per unit dose.

9. The consumable product of claim 1, wherein the probiotic is present in an amount of about 3 billion colony-forming units per unit dose.

10. The consumable product of claim 1, further comprising a natural preservative.

11. The consumable product of claim 10, wherein the natural preservative comprises rosemary extract, sorbic acid, mixed tocopherols, green tea extract, or a combination thereof.

12. The consumable product of claim 11, wherein the consumable product comprises from about 1% to about 3% by volume of liquid consumable product or from about 1% by 3% by weight of solid consumable product.

13. The consumable product of claim 11, wherein the natural preservative comprises rosemary extract.

14. The consumable product of claim 1, wherein the consumable product does not include any artificial preservatives.

15. The consumable product of claim 1, wherein the product further comprises a prebiotic.

16. The consumable product of claim 15, wherein the prebiotic comprises pectin, β-glucan, guar gum, methylcellulose, psyllium fiber, resistant starch, fructooligosaccharides, beet pulp, or any combination thereof.

17. The consumable product of claim 1, wherein the consumable product further comprises a natural flavor.

18. The consumable product of claim 17, wherein the natural flavor comprises bacon, chicken, turkey, duck, lamb, beef liver, chicken liver, dairy, salmon, tuna, scallop, shrimp, dried brewer's yeast, cinnamon, peanut butter, apples, blueberries, sweet potato, molasses, tapioca syrup, dried milk, dried egg whites, or any combination thereof.

19. The consumable product of claim 1, wherein the consumable product further comprises a texture-modifying agent.

20. The consumable product of claim 19, wherein the texture-modifying agent comprises glycerin, polysorbate 80, flaxseed or flaxseed oil, medium chain triglyceride oil, oat flour, palm fruit oil, powdered cellulose, white rice flour, brown rice flour, rice bran oil, soybean oil, sunflower lecithin, coconut oil, rolled oats, potato starch, peas, gum arabic, sodium alginate, salmon oil, anchovy oil, sardine oil, or another fish oil, wheat germ oil, malic acid, propylene glycol, calcium sulfate dehydrate, dextrin, purified water, or any combination thereof.

21. The consumable product of claim 1, wherein the consumable product further comprises at least one additional active ingredient.

22. The consumable product of claim 21, wherein the at least one additional active ingredient comprises glucosamine hydrochloride, methylsulfonylmethane, chondroitin sulfate, fish oil, chamomile, passionflower, ginger, L-tryptophan, turmeric, Boswellia, hempseed powder, hempseed oil, bone broth, collagen, milk thistle extract, chamomile, lemon balm, valerian, turmeric, tea tree oil, slippery elm, aloe vera, dandelion, S-adenosylmethionine, L-theanine, seaweed, chlorella, β-carotene, taurine, a vitamin, a mineral, an electrolyte, or any combination thereof.

23. The consumable product of claim 1, wherein the consumable product comprises:
a. cannabidiol in an amount such that about 1 mg to about 5 mg are administered per 10 pounds of pet body weight;
b. a probiotic comprising about 1 to about 10 billion colony forming units of Bacillus coagulans GBI-30, 6086 or another Bacillus coagulans strain; Lactobacillus acidophilus; Lactobacillus plantarum; Lactobacillus brevis; Lactobacillus fermentum; Lactobacillus lactis; or any combination thereof; and
c. pumpkin in an amount such that about 3 mg are administered per 10 pounds of pet body weight.

24. The consumable product of claim 1, wherein the pet is a cat, a dog, a horse, or a bird.

25. A method for treating, preventing, or reducing at least one symptom of a disease or condition in a pet, the method comprising administering the consumable product of claim 1 to the pet.

26. The method of claim 25, wherein the disease or condition comprises cancer, osteoarthritis, epilepsy, inflammatory bowel disease, dental disease, cardiovascular disease, diabetes, obesity, mood disorders, autoimmune disease, yeast infection, bacterial infection, viral infection, parasitic infection, anxiety, dysbiosis, allergies, food sensitivities, asthma, or a combination thereof.

27. The method of claim 25, wherein at least one symptom or the at least one side effect comprises pain, appetite loss, noise phobia, nausea, inflammation, diarrhea, constipation, gas, bloating, bad breath, leaky gut, a nutrient deficiency, stress, or a combination thereof.

28. The method of claim 25, further comprising co-administering the consumable product with at least one other medication effective against the disease or condition, wherein the at least one other medication comprises an antibiotic, a chemotherapy drug, a steroid, an antifungal medication, an anti-parasitic agent, a painkiller or analgesic, an antidepressant, an antihistamine, an immunosuppressive agent, an anticonvulsant, an appetite stimulant, an antiemetic, an appetite suppressant, or a combination thereof.

29. The method of claim 28, wherein the consumable product and the at least one other medication are co-formulated.

30. A method for reducing or preventing at least one side effect of a medication on a pet, the method comprising administering the consumable product of claim 1 to the pet.
